(19) United States
(12) Patent Application Publication
(10) Pub. No.: US 2021/0236688 A1
(43) Pub. Date: Aug. 5, 2021

(54) Biodegradable Metallic - Polymeric Composite Prosthesis for Heart Valve Replacement

(71) Applicants: University of Pittsburgh - Of the Commonwealth System of Higher Education, Pittsburgh, PA (US); Ri.MED Foundation, Palermo (IT); University of Cincinnati, Cincinnati, OH (US)

(72) Inventors: William R. Wagner, Gibsonia, PA (US); Antonio D'Amore, Pittsburgh, PA (US); Vesselin N. Shanov, Cincinnati, OH (US)

(21) Appl. No.: 17/050,538

(22) PCT Filed: Apr. 25, 2019
(86) PCT No.: PCT/US2019/029121

§ 371 Date: Oct. 26, 2020

Related U.S. Application Data
(60) Provisional application No. 62/663,721, filed on Apr. 27, 2018.

(51) Int. Cl. A61L 27/04; A61F 2/24; A61L 27/18; A61L 27/58; A61L 27/34; A61L 33/06; A61L 33/00; C08L 75/04; C08L 65/04

(52) U.S. Cl. CPC A61L 27/047; A61F 2/2415; A61F 2/2418; A61L 27/18; C08L 65/04; A61L 27/34; A61L 33/068; A61L 33/0023; C08L 75/04; A61L 27/58

(57) ABSTRACT

Provided herein is a prosthetic heart valve device including a biocompatible and biodegradable metal frame comprising a proximal end, a distal end, and a sidewall therebetween, the sidewall having a plurality of openings therethrough. The device further includes a biocompatible and biodegradable polymeric heart valve having an annular portion attached at least one contact point to the proximal end of the frame and at least one leaflet attached to and extending distally from the annular portion.

### CROSS-REFERENCE TO RELATED APPLICATIONS

The present application claims priority to U.S. Provisional Patent Application No. 62/663,721, filed Apr. 27, 2018, the contents of which are incorporated herein by reference in their entirety.

### STATEMENT REGARDING FEDERAL FUNDING

This invention was made with government support under Grant Number EEC-0812348, awarded by the National Science Foundation. The government has certain rights in the invention.

### BACKGROUND

### Field of the Invention

Provided herein are prosthetic heart valves, more particularly, prosthetic heart valves including biodegradable metal-polymer composites.

### Description of Related Art

Valvular heart disease remains a global management challenge of access to optimal therapy, especially in children as well as in developing countries where rheumatic disease remains prevalent.

Current valve replacement technologies fall into two major categories: metallic valves and bioprosthetic valves. While generally effective, these approaches suffer from a number of limitations. More specifically, metallic valves benefit from excellent durability but they come with the burden of anticoagulation therapy. On the other hand, bioprosthetic valves do not require anticoagulant therapy, but do not exhibit the same durability with a significant fraction (e.g. 5-30%) of the implanted devices exhibiting structural deterioration within five years from implantation. These limitations have propelled the interest for the tissue engineering approach.

### SUMMARY

The ideal valve prosthesis provides for non-thrombogenic long term durability and the potential to adapt to somatic growth. A potential solution is a tissue engineered heart valve (TEHV).

Accordingly, provided herein is a prosthetic heart valve device including a biocompatible and biodegradable metal frame comprising a proximal end, a distal end, and a sidewall therebetween, the sidewall having a plurality of openings therethrough; and a biocompatible and biodegradable polymeric heart valve including an annular portion attached by at least one contact point to the proximal end of the frame and at least one leaflet attached to and extending distally from the annular portion.

Further embodiments or aspects are set forth in the following numbered clauses:

Clause 1: A heart valve device comprising: a substantially cylindrical frame of a biodegradable metal, having a wall with a plurality of openings, such as an array of diamond and hexagonal shape openings, in the wall along a central axis, e.g., of 18 mm, and a diameter, e.g., of 22 mm; and a polymeric heart valve comprising an annular portion attached to the frame and a plurality of leaflets attached to and extending from the annular portion of the polymeric heart valve and connected to each other, e.g., at a portion of their periphery adjacent to the attachment point of the leaflets to the annular portion.

Clause 2: The device of clause 1, wherein the frame comprises, or is made from, a biodegradable metal selected from magnesium, zinc, iron, or alloys thereof.

Clause 3: The device of clause 1 or clause 2, wherein the biodegradable metal is a magnesium alloy.

Clause 4: The device of any of clauses 1-3, wherein the magnesium alloy is AZ31, such as an alloy comprising or consisting of: 2.5-3.5%, e.g., 3% Al; 0.7-1.3%, e.g., 1% Zn; optionally 0.2% (min), e.g., 0.3% Mn; and Mg (balance).

Clause 5: The device of any of clauses 1-4, wherein the magnesium alloy is AZ61.

Clause 6: The device of any of clauses 1-5, wherein the magnesium alloy is AZ91.

Clause 7: The device of any of clauses 1-6, wherein the magnesium alloy is WE43, such as an alloy comprising or consisting of: 4 wt % yttrium, 2.3 wt % neodymium, 1 wt % other rare earth elements, 0.6 wt % zirconium, and magnesium (balance).

Clause 8: The device of any of clauses 1-7, wherein the magnesium alloy comprises or consists of: magnesium, yttrium, calcium, and zirconium.

Clause 9: The device of any of clauses 1-8, wherein the polymeric heart valve comprises a porous matrix of a biodegradable polymer composition.

Clause 10: The device of any of clauses 1-9, wherein the polymeric heart valve comprises a porous matrix of fibers of a biodegradable polymer composition.

Clause 11: The device of any of clauses 1-10, wherein one or more of the leaflets and/or the annular portion of the polymeric heart valve comprises an anisotropic matrix of fibers of the biodegradable polymer composition.

Clause 12: The device of any of clauses 1-11, wherein one or more of the leaflets and/or the annular portion of the polymeric heart valve comprises electrospun fibers of the biodegradable polymer composition.

Clause 13: The device of any of clauses 1-12, wherein the polymeric heart valve comprises two or three leaflets.

Clause 14: The device of any of clauses 1-13, wherein the frame is balloon-expandable, and has a compressed state and an expanded state, wherein the diameter of the frame in its compressed state is 50% or less than the diameter of the frame in its expanded state.

Clause 15: The device of any of clauses 1-14, wherein the frame is balloon-expandable, and has a compressed state and an expanded state, wherein the diameter of the frame in its compressed state fits within a catheter sheath.

Clause 16: The device of any of clauses 1-15, wherein the polymeric heart valve has a height along a central axis, and a diameter, and the height of the frame ranges from 50% to 150% of the height of the polymeric heart valve.

Clause 17: The device of any of clauses 1-16, further comprising an antithrombogenic coating on the frame and/or on the polymeric heart valve.

Clause 18: The device of any of clauses 1-17, wherein the antithrombogenic coating is a polymer composition comprising a polyurethane with an antithrombogenic group.

Clause 19: The device of any of clauses 1-18, wherein the antithrombogenic group is a zwitterionic group, such as phosphorylcholine (PC) or a betaine, e.g., sulfobetaine, or carboxybetaine.

Clause 20: A method of manufacturing a heart valve device comprising attaching to a substantially cylindrical frame of a biodegradable metal, having a wall with a plurality of openings in the wall, a height along a central axis, and a diameter, a polymeric heart valve comprising an annular portion and a plurality of leaflets attached to and extending from the annular portion of the polymeric heart valve and connected to each other, e.g., at a portion of their periphery adjacent to the attachment point of the leaflets to the annular portion.

Clause 21: The method of clause 20, wherein the frame comprises or is made from a biodegradable metal selected from magnesium, zinc, iron, or alloys thereof.

Clause 22: The method of clause 20 or clause 21, wherein the biodegradable metal is a magnesium alloy.

Clause 23: The method of any of clauses 20-22, wherein the magnesium alloy is AZ31, such as an alloy comprising or consisting of: 2.5-3.5%, e.g., 3% Al; 0.7-1.3%, e.g., 1% Zn; optionally 0.2% (min), e.g., 0.3% Mn; and Mg (balance).

Clause 24: The method of any of clauses 20-23, wherein the magnesium alloy is WE43, such as an alloy comprising or consisting of: 4 wt % yttrium, 2.3 wt % neodymium, 1 wt % other rare earth elements, 0.6 wt % zirconium, and magnesium (balance).

Clause 25: The method of any of clauses 20-24, wherein the magnesium alloy comprises or consists of: magnesium, yttrium, calcium, and zirconium.

Clause 26: The method of any of clauses 20-25, wherein the magnesium alloy is AZ61.

Clause 27: The method of any of clauses 20-27, wherein the magnesium alloy is AZ91.

Clause 28: The method of any of clauses 20-27, wherein the polymeric heart valve comprises a porous matrix of a biodegradable polymer composition.

Clause 29: The method of any of clauses 20-28, wherein the polymeric heart valve comprises a porous matrix of fibers of a biodegradable polymer composition.

Clause 30: The method of any of clauses 20-29, wherein one or more of the leaflets and/or the annular portion of the polymeric heart valve comprises an anisotropic matrix of fibers of the biodegradable polymer composition.

Clause 31: The method of any of clauses 20-30, wherein one or more of the leaflets and/or the annular portion of the polymeric heart valve comprises electrospun fibers of the biodegradable polymer composition.

Clause 32: The method of any one of claims 20-31, wherein the polymeric heart valve comprises two or three leaflets.

Clause 33: The method of any of clauses 20-32, wherein the frame is balloon-expandable, and has a compressed state and an expanded state, wherein the diameter of the frame in its compressed state is 50% or less than the diameter of the frame in its expanded state.

Clause 34: The method of any of clauses 20-33, wherein the frame is balloon-expandable, and has a compressed state and an expanded state, wherein the diameter of the frame in its compressed state fits within a catheter sheath.

Clause 35: The method of any of clauses 20-34, wherein the polymeric heart valve has a height along a central axis, and a diameter, and the height of the frame ranges from 50% to 150% of the height of the polymeric heart valve.

Clause 36: The method of any of clauses 20-35, further comprising an antithrombogenic coating on the frame or on the polymeric heart valve.

Clause 37: The method of any of clauses 20-36, wherein the antithrombogenic coating is a polymer composition comprising a polyurethane with an antithrombogenic group.

Clause 38: The method of any of clauses 20-37, wherein the antithrombogenic group is a zwitterionic group, such as phosphorylcholine (PC) or a betaine, e.g., sulfobetaine, or carboxybetaine.

Clause 39: A method of repairing a heart valve in a patient, comprising placing the device of any of clauses 1-19 in a heart valve annulus of the patient.

Clause 40: The method of clause 39, wherein the heart valve device is expanded in place in the valve annulus of the patient using a balloon catheter.

Clause 41: The method of clause 39 or 40, wherein the device is delivered percutaneously or transapically.

Clause 42: An annuloplasty ring comprising an annular or c-shaped member of a biodegradable metal retained within an annular or c-shaped sheath of a bioerodable polymer.

Clause 43: An annuloplasty method, comprising implanting the annuloplasty ring of clause 42 in a heart valve annulus of a patient.

Clause 44: A prosthetic heart valve device comprising: a biocompatible and biodegradable metal frame comprising a proximal end, a distal end, and a sidewall therebetween, the sidewall comprising a plurality of openings therethrough; and a biocompatible and biodegradable polymeric heart valve comprising an annular portion attached by at least one contact point to the proximal end of the frame and at least one leaflet attached to and extending distally from the annular portion.

Clause 45: The device of clause 44, wherein the frame comprises a magnesium (Mg) alloy.

Clause 46: The device of clause 44 or clause 45, wherein the Mg alloy comprises aluminum (Al), Zinc (Zn), and, optionally, manganese (Mn).

Clause 47: The device of any of clauses 44-46, wherein the Mg alloy is AZ31.

Clause 48: The device of any of clauses 44-47, wherein the Mg alloy is AZ61.

Clause 49: The device of any of clauses 44-48, wherein the Mg alloy is AZ91.

Clause 50: The device of any of clauses 44-49, wherein the Mg alloy comprises yttrium (Y), neodymium (Nd), and zirconium (Zr).

Clause 51: The device of any of clauses 44-50, wherein the Mg alloy is WE43.

Clause 52: The device of any of clauses 44-51, wherein the biocompatible and biodegradable polymer comprises a polyurethane.

Clause 53: The device of any of clauses 44-52, wherein the polyurethane is a polycarbonate urethane urea (PCUU).

Clause 54: The device of any of clauses 44-53, wherein the polymeric heart valve comprises two or three leaflets.

Clause 55: The device of any of clauses 44-54, further comprising a polymeric coating on at least a portion of the device.

Clause 56: The device of any of clauses 44-55, wherein the polymeric coating is a porous, non-biodegradable coating, optionally a poly(p-xylylene) polymer, optionally one or more of parylene C, parylene N, parylene D, parylene F, and a halogen-free parylene.

Clause 57: The device of any of clauses 44-56, wherein the non-degradable coating has a thickness of 10 nm-10 μm, optionally 80 nm-300 nm, optionally 80 nm-100 nm.

Clause 58: The device of any of clauses 44-57, wherein the polymeric coating is a biodegradable coating.

Clause 59: The device of any of clauses 44-58, wherein the biodegradable coating has a thickness of up to 10 μm.

Clause 60: The device of any of clauses 44-59, wherein the coating is an antithrombogenic coating.

Clause 61: The device of any of clauses 44-60, wherein the antithrombogenic coating is a polymer composition comprising a polyurethane with an antithrombogenicgroup.

Clause 62: The device of any of clauses 44-61, wherein the antithrombogenic group is a phosphorylcholine or a betaine.

Clause 63: A method of manufacturing a heart valve device comprising attaching an annular portion of a biocompatible and biodegradable polymeric heart valve comprising an annular portion and a leaflet extending therefrom to a proximal end of a biocompatible, biodegradable metal frame having a proximal end, a distal end, and a sidewall therebetween, the sidewall comprising a plurality of openings therethrough.

Clause 64: The method of clause 63, wherein the frame comprises a Mg alloy.

Clause 65: The method of clause 63 or clause 64, wherein the Mg alloy comprises Al, Zn, and, optionally, Mn.

Clause 66: The method of any of clauses 63-65, wherein the Mg alloy is AZ31.

Clause 67: The method of any of clauses 63-66, wherein the Mg alloy is AZ61.

Clause 68: The method of any of clauses 63-67, wherein the Mg alloy is AZ91.

Clause 69: The method of any of clauses 63-68, wherein the Mg alloy comprises Y, Nd, and Zr.

Clause 70: The method of any of clauses 63-69, wherein the Mg alloy is WE43.

Clause 71: The method of any of clauses 63-70, wherein the biocompatible and biodegradable polymer comprises a polyurethane.

Clause 72: The method of any of clauses 63-71, wherein the polyurethane is a polycarbonate urethane urea (PCUU).

Clause 73: The method of any of clauses 63-72, wherein the polymeric heart valve comprises two or three leaflets.

Clause 74: The method of any of clauses 63-73, further comprising a polymeric coating on at least a portion of the device.

Clause 75: The method of any of clauses 63-74, wherein the polymeric coating is a porous, non-biodegradable coating, optionally a poly(p-xylylene) polymer, optionally one or more of parylene C, parylene N, parylene D, parylene F, and a halogen-free parylene.

Clause 76: The method of any of clauses 63-75, wherein the non-degradable coating has a thickness of 10 nm-10 μm, optionally 80 nm-300 nm, optionally 80 nm-100 nm.

Clause 77: The method of any of clauses 63-76, wherein the polymeric coating is a biodegradable coating.

Clause 78: The method of any of clauses 63-77, wherein the biodegradable coating has a thickness of up to 10 μm.

Clause 79: The method of any of clauses 63-78, wherein the coating is an antithrombogenic coating.

Clause 80: The method of any of clauses 63-79, wherein the antithrombogenic coating is a polymer composition comprising a polyurethane with an antithrombogenic group.

Clause 81: The method of any of clauses 63-80, wherein the antithrombogenic group is a phosphorylcholine or a betaine.

Clause 82: A method of repairing a heart valve in a patient, comprising placing the device of any of clauses 44-62 in a heart valve annulus of the patient.

Clause 83: The method of clause 82, wherein the heart valve device is expanded in place in the heart valve annulus of the patient using a balloon catheter.

Clause 84: The method of clause 82 or clause 83, wherein the device is delivered percutaneously or transapically.

Clause 85: A kit comprising a catheter and the device of any of clauses 44-62.

Clause 86: A prosthetic heart valve device comprising: an AZ31 Mg alloy, substantially cylindrical frame comprising a proximal end, a distal end, and a sidewall therebetween, the sidewall comprising a plurality of openings therethrough; a PCUU heart valve comprising an annular portion attached by at least one contact point to the proximal end of the frame and two or three leaflets attached to and extending distally from the annular portion; and a polymeric coating on at least a portion of the frame.

Clause 87: A prosthetic heart valve device comprising: a WE43 Mg alloy, substantially cylindrical frame comprising a proximal end, a distal end, and a sidewall therebetween, the sidewall comprising a plurality of openings therethrough; a PCUU heart valve comprising an annular portion attached by at least one contact point to the proximal end of the frame and two or three leaflets attached to and extending distally from the annular portion; and a polymeric coating on at least a portion of the frame.

### BRIEF DESCRIPTION OF THE DRAWINGS

FIG. 1. A) Mg frame for transcatheter heart valve device fabricated by photo-chemical etching mounted on biodegradable polymeric valve, with the arrow representing the central axis of the device; B) ventricular view, with the double-sided arrow representing the diameter of the device; C) arterial view; and D) stent without valves.

FIG. 2. Schematic illustrating the manufacturing steps in the photo-chemical etching approach for making a Mg frame.

FIGS. 3A and 3B depict schematically an example of a balloon catheter for use in percutaneous deployment of one aspect of the heart valve device described herein. FIG. 3A depicts the heart valve device as it is delivered, within the sheath of a catheter. FIG. 3B depicts the heart valve device extending from the distal end of the catheter sheath, as it is pressed in place by the balloon in a heart valve annulus in a patient.

FIG. 4. A) Ventricular side: Mg stent with polymeric valve explants, in situ. B) Mg stent with polymeric valve explants, removed from the right ventricular outflow track (RVOT). C) 2D epicardial echocardiography after TEHV implant (arrow indicates stent housing in the native pulmonary artery). D) Flow Doppler showing antegrade flow through the TEHV prosthesis in the pulmonary position (no observed regurgitation). E) Continuous-wave Doppler demonstrating forward flow through the pulmonary prosthesis. F-G) arterial side and ventricular side explants (respectively) showing no evidence of thrombus formation. H) Mg stent SEM showing metal degradation after blood exposure in vivo.

FIGS. 5A-5C show useful dimensions and processing of a frame as described in the examples below.

FIG. 6 shows an intraoperative photograph of the scaffold-based pulmonary heart valve being sutured in place with 4-0 polypropylene suture after excision of native pulmonary valve.

FIG. 7 shows A) Gross explant of PCUU pulmonary valve on Mg degradable stent following 12-hour in vivo study. B) PCUU valve leaflets after removal from Mg stent prior to washing and histological fixation. No gross thrombosis identified on PCUU leaflets. C) Mg stent with fibrin sheath formation after explant following 12 hour in vivo functional assessment. There was no identified obstruction of the valve or outflow tract due to this finding.

FIG. 8 shows A) Hematoxylin and eosin staining of an explanted PCUU valve leaflet. The PCUU leaflet is functionally intact without active cellular uptake. B) PCUU leaflet with SEM shows no change in fibrous/porous structure of the leaflet, and no platelet activation or microthrombus. C) Mg AZ 31 alloy stent shown with SEM displays evidence of early surface oxidation, but no clear bulk degradation.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the singular form of “a”, “an”, and “the” include plural referents unless the context clearly dictates otherwise.

As used herein, the terms “upper”, “lower”, “right”, “left”, “vertical”, “horizontal”, “top”, “bottom”, “lateral”, “longitudinal”, and derivatives thereof, shall relate to the invention as it is oriented in the drawing figures. The term “proximal” refers to a portion of a structure nearest to the center of the structure or to a point of attachment or actuation of the structure. For example, a “proximal portion” of a syringe is the portion of the syringe configured to be grasped by a user. The term “distal” refers to a portion of a structure farthest away from the center or from the point of attachment or actuation of the structure (e.g., the portion of the structure opposite from the proximal portion). For example, a “distal portion” of a syringe is the end of the syringe including the needle or nozzle. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

As used herein, the “treatment” or “treating” of a condition, wound, or defect means administration to a patient by any suitable dosage regimen, procedure and/or administration route of a composition, device or structure with the object of achieving a desirable clinical/medical end-point, including repair and/or replacement of a bicuspid or mitral valve.

As used herein, the term “patient” or “subject” refers to members of the animal kingdom including but not limited to human beings and “mammal” refers to all mammals, including, but not limited to human beings.

A metal, metal alloy, and/or polymer composition is “biocompatible” in that the material and, where applicable, degradation products thereof, are substantially non-toxic to cells or organisms within acceptable tolerances, including substantially non-carcinogenic and substantially non-immunogenic, and are cleared or otherwise degraded in a biological system, such as an organism (patient) without substantial toxic effect. Non-limiting examples of degradation mechanisms within a biological system include chemical reactions, hydrolysis reactions, and enzymatic cleavage. In addition, given one intended use of a prosthetic valve device as described herein, “biocompatible” means that the material, and degradation products thereof, are substantially or completely non-thrombogenic.

A“metal” includes pure metals, such as pure magnesium, zinc, or iron, or alloys thereof, e.g., as described below.

As used herein, the term “heart valve” means a valve structure in the heart comprising heart valve leaflet(s) disposed within an annulus. The term includes any number of leaflets, including without limitation monoleaflet, bileaflet, and trileaflet valves, including the aortic, pulmonary, mitral, and bicuspid valves.

As used herein, the term “polymer composition” is a composition comprising one or more polymers. As a class, “polymers” includes, without limitation, homopolymers, heteropolymers, co-polymers, block polymers, block co-polymers and can be both natural and/or synthetic. Homopolymers contain one type of building block, or monomer, whereas copolymers contain more than one type of monomer. The term “(co)polymer” and like terms refer to either homopolymers or copolymers.

A polymer “comprises” or is “derived from” a stated monomer if that monomer is incorporated into the polymer. Thus, the incorporated monomer (monomer residue) that the polymer comprises is not the same as the monomer prior to incorporation into a polymer, in that at the very least, certain groups/moieties are missing and/or modified when incorporated into the polymer backbone. A polymer is said to comprise a specific type of linkage if that linkage is present in the polymer.

As described herein, a “fiber” is an elongated, slender, thread-like and/or filamentous structure. A “matrix” is any two- or three-dimensional arrangement of a composition, e.g., a porous structure that optionally comprises elements (e.g., fibers), either ordered (e.g., in a woven or non-woven mesh) or randomly-arranged (as is typical with a mat of fibers typically produced by electrospinning) and can be isotropic or anisotropic. As indicated herein, a porous matrix is a matrix comprising a plurality of pores or interstices, such as a porous hydrogel comprising pores created by the dissolution of a porogen from a solid hydrogel, or openings between fibers deposited in a woven or non-woven mesh.

As used herein, the term “porosity” refers to a ratio between a volume of all the pores within the polymer composition and a volume of the whole polymer composition. For instance, a polymer composition with a porosity of 85% would have 85% of its volume containing pores and 15% of its volume containing the polymer. In certain non-limiting embodiments, the porosity of the structure, once to porogen dissolves, is at least 60%, 65%, 70%, 75%, 80%, 85%, or 90%, or increments therebetween.

By “biodegradable or “bioerodable”, it is meant that a polymer, metal, or metal alloy, once implanted and placed in contact with bodily fluids and tissues, will degrade either partially or completely through chemical reactions with the bodily fluids and/or tissues, typically and often preferably over a time period of hours, days, weeks or months. Non-limiting examples of such chemical reactions for polymers include acid/base reactions, hydrolysis reactions, and enzymatic cleavage. The biodegradation rate of the polymer matrix may be manipulated, optimized or otherwise adjusted so that the matrix degrades over a useful time period. The polymer or polymers typically will be selected so that it degrades in situ over a time period to optimize tissue regeneration in its use. Metals typically degrade by corrosion by chemical or electrochemical reactions.

As used herein, the terms “therapeutic agent” and “therapeutic agents” refer to any composition(s), such as drug(s) or active agent(s) having a preventative or therapeutic effect, including and without limitation, antibiotics, peptides, hormones, organic molecules, vitamins, supplements, factors, proteins and chemoattractants. A “therapy” or “treatment” refers to administration of a therapeutic composition, such as the compositions described herein, in amounts effective to reach an acceptable end point, e.g., a clinical end point, such as the repair of a heart valve.

As used herein, the terms “cell” and “cells” refer to any types of cells from any animal, such as, without limitation, rat, mice, monkey, and human. For example and without limitation, cells can be progenitor cells, such as stem cells, or differentiated cells, such as endothelial cells, or smooth muscle cells. In certain embodiments, cells for medical procedures can be obtained from the patient for autologous procedures or from other donors for allogeneic procedures.

Provided herein is a prosthetic heart valve device. The device includes a biocompatible, biodegradable frame and a biocompatible, biodegradable valve portion. The frame can be of any suitable shape, and, in non-limiting embodiments or aspects, the frame is substantially cylindrical or cylindrical. As will be described more thoroughly below, the frame can be comprised of a biocompatible, biodegradable metal or metal alloy. In non-limiting embodiments or aspects, the frame can include a proximal end, a distal end, and a sidewall therebetween. In non-limiting embodiments or aspects, the sidewall can include one or more openings therethrough. In this way, the frame can take the form of a stent.

As noted above, the frame can have any suitable shape. In addition, the frame may have any useful diameter. In non-limiting embodiments or aspects, the diameter of the frame is sized to fit into a valve annulus, such as a mitral (bicuspid) valve annulus or a tricuspid valve annulus. For a human or animal of 70 kg-120 kg, an exemplary diameter range for the frame for an aortic or pulmonary position (e.g.) is from 15 mm to 32 mm, e.g., from 17 mm to 30 mm, for example and without limitation, 17 mm, 23 mm, or 30 mm; an exemplary diameter range for the frame for a mitral valve is from 20 mm to 40 mm, e.g., from 24 mm to 34 mm, for example and without limitation, 24 mm, 30 mm, or 34 mm; an exemplary diameter range for the frame for a tricuspid valve is from 25 mm to 35 mm, e.g., from 26 mm to 34 mm, for example and without limitation, 26 mm, 30 mm, or 34 mm; an exemplary diameter/height (d/h, ratio of the diameter to the longitudinal length of the frame) ranges from 1 to 1.5, e.g., from 1.1 to 1.3, for example 1.285; and the minimum access vessel is, e.g., approximately 5.5 mm to 6 mm, corresponding to 18 F (French) to 21 F.

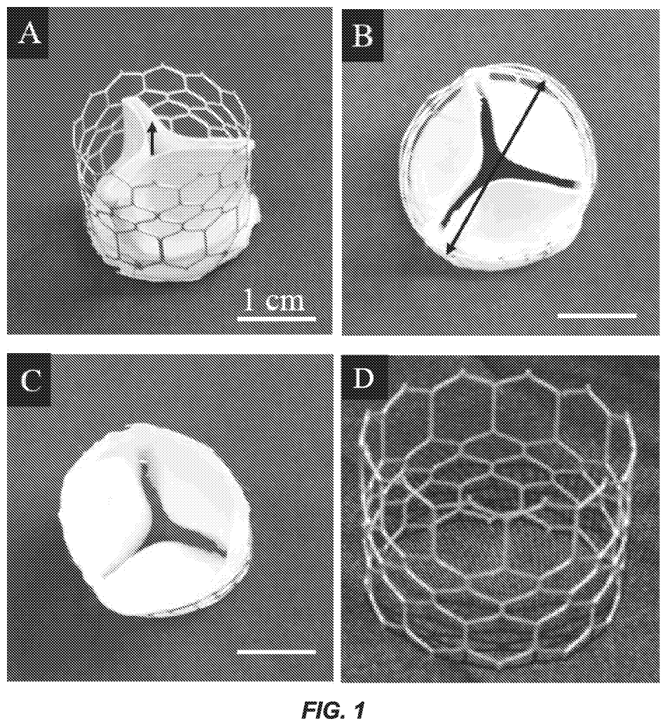

The valve of the device can be formed of a biocompatible, biodegradable polymer, useful examples of which are provided in detail below. The valve includes an annular portion that can be attached to the frame portion by suitable methods, such as by suturing, annealing, or other known methods of attaching a polymer to a metal or metal alloy. In non-limiting embodiments or aspects, the annular portion of the valve is attached to the frame at a proximal or distal end thereof (for example, and without limitation, as shown in FIG. 1). In non-limiting embodiments or aspects, the valve extends away from (either proximally or distally) from the attachment point(s) between the valve and the frame, for example as also shown in FIG. 1. In non-limiting embodiments or aspects, the valve extends through an interior of the frame. The valve can be any useful shape or configuration, however, in non-limiting embodiments or aspects the valve is a monoleaflet valve, a bileaflet valve, or a trileaflet valve. In non-limiting embodiments or aspects, in addition to the valve, the device includes a skirt of a biocompatible, biodegradable material, arranged about the circumference of the frame, for example to reduce or prevent leakage.

Because the frame is biocompatible and biodegradable, existing self-expanding, superelastic materials, such as nitinol, are not suitable. Accordingly, in embodiments or aspects where expansion of the device is required, such as where the device is delivered by a percutaneous, transapical, or another minimally-invasive route, the cylinder is balloon-expandable from a compressed state to an expanded state with a larger diameter than the diameter of the compressed state. In aspects, the cylindrical frame is expandable using a balloon of a balloon catheter device, as are known in the expandable stent and balloon catheter fields. For use in valve repair, the frame should have sufficient strength to resist buckling, and may be supplemented with a non-degradable annuloplasty ring as is broadly-known, or a biodegradable annuloplasty ring as described below—though typically annuloplasty is performed in open-heart or other minimally-invasive procedure, but not through femoral or other remote percutaneous access methods. Because the frame can be balloon-expanded, in aspects, the metallic biodegradable stent will be designed with yield stress appropriate to allow plastic deformation upon expansion using a balloon catheter.

In aspects, the device is delivered transapically or by open-heart procedures. In a transapical procedure, larger sheath (catheter, cannula, or trocar) can be used, and as such, the frame cylinder need not be compressed as much as would be necessary during a femoral or other percutaneous, peripheral approach. As such for a transapical approach the amount of frame expansion or dilation necessary to deliver the device may be minimized, making additional alloys, such as stronger, but less flexible or higher yield stress alloys available for use. In open-heart procedures, the device need not be expanded, and as such the choice of biodegradable metal is further broadened to include non-expandable metals, such that optimal strength may be favored over expandability. As described below, there are a number of suitable alloys, with choice of the alloy for each use being within the abilities of a person of ordinary skill in the art.

For such methods as described herein, in non-limiting embodiments or aspects, a kit is provided, the kit including a prosthetic heart valve as described herein, a delivery catheter, and, optionally, a guidewire, introducer sheath, and/or any other suitable device for introducing a device into, for example and without limitation, the annulus of a heart valve.

As described above, the frame is manufactured from a biocompatible, biodegradable metal, which corrodes in vivo. Examples of biodegradable metals can be found generally in Hermawan, Biodegradable Metals: From Concept to Applications, Springer Brief in Materials (2012), but, briefly, include metals and alloys of metals including magnesium (Mg), aluminum (Al), yttrium (Y), lithium (Li), calcium (Ca), manganese (Mn), zinc (Zn), iron (Fe), or neodymium (Nd). Mg and alloys thereof, Zn and alloys thereof, and Fe and alloys thereof are examples of materials that are recognized as being suited for use in biodegradable stents (See, e.g., Hermawan et al., Developments in Metallic Biodegradable Stents, Acta Biomaterialia 6 (2010) 1693-1697). Iron and iron-manganese materials have been used as biodegradable metals. Zinc (Zn) and Zn alloys, including Mg—Zn alloys also have found use in degradable devices (See. e.g., Yang et al., Evolution of the degradation mechanism of pure Zn stent in the one-year study of rabbit abdominal aorta model, Biomaterials 145 (2017) 92-105). Mg and Mg alloy materials are useful as biodegradable metals, as disclosed herein, and include, for example, a Mg foil (including Mg or an alloy thereof) with a composition including but not limited to pure Mg, or a Mg alloy with any useful composition, such as from the AZ series, which can be non-doped or doped with rare earth elements (in non-limiting examples these can include light rare earth elements, such as lanthanum (La), cerium (Ce), praseodymium (Pr), Nd, promethium (Pm), samarium (Sm), europium (Eu), and gadolinium (Gd) or heavy rare earth elements such as terbium (Tb), dysprosium (Dy), holmium (Ho), erbium (Er), thulium (Tm), ytterbium (Yb), lutetium (Lu), and Y), and including other elements such as lithium (Li), Ca, zirconium (Zr), silicon (Si), nickel (Ni), and/or copper (Cu). AZ series of Mg alloys include alloys of Mg, Al, and Zn, such as AZ31-type alloys, which comprise (in mass %), for example: 2.5-3.5%, e.g., 3% Al; 0.7-1.3%, e.g., 1% Zn; optionally 0.2% (min), e.g., 0.3% Mn; and Mg (balance), for example 3% Al and 1% Zn, balanced with pure Mg. In one aspect, the frame is manufactured from an AZ31-type alloy. Alternatively, AZ61 and AZ91 Mg alloys can be also employed for this application. Another useful Mg alloy is WE43 with a nominal composition of, e.g., 4 wt % Y, 2.3 wt % Nd, 1 wt % other rare earth elements, 0.6 wt % Zr, and Mg (balance).

Where alloys are described in terms of percentage by weight, it is assumed that the alloys may comprise some unspecified, but nominal, amount of impurities due to production. Thus, as an example, an alloy consisting of 3% Al; 1% Zn; and Mg (balance) also may include any impurities due to production.

United States Patent Application Publication No. 2018/0100219, the disclosure of which is incorporated herein by reference, describes further useful biodegradable metal alloys with improved physical qualities, such as strength and ductility, e.g., Mg alloys, comprising, e.g., Mg, with non-toxic and effective amounts of Y, Ca, Zr, Zn, Al, Ce, and/or silver (Ag), for example Mg with Y, Ca, and Zr. The Mg alloys described therein can be “tuned” to optimize corrosion rate. In aspects, aluminum (Al) is present in an amount of from about 1.0 to 9.0 weight percent based on total weight of the composition. In other aspects, the Al is present in an amount of about 2.0 weight percent based on total weight of the composition. In aspects, Mn is present in an amount of from about 0.1 to about 1.0 weight percent based on total weight of the composition. In other aspects, the Mn is present in an amount of about 0.2 weight percent based on total weight of the composition. In aspects, Ag is present in an amount of from about 0.25 to about 1.0 weight percent based on total weight of the composition. In other aspects, the Ag is present in an amount of about 0.25 weight percent based on total weight of the composition. In aspects, Ce is present in an amount of from about 0.1 to about 1.0 weight percent based on total weight of the composition. In other aspects, the Ce is present in an amount of about 0.5 weight percent based on total weight of the composition. In certain aspects, strontium (Sr) is present in an amount of from about 1.0 to about 4.0 weight percent based on total weight of the composition. In other aspects, the Sr can be present in an amount of about 3.0 weight percent.

Examples of the Mg alloys of United States Patent Application Publication No. 2018/0100219, useful for manufacturing the frame include, for example and without limitation, biodegradable, metal alloys:
consisting of from about 1.0 weight percent to about 6.0 weight percent of Zn; from greater than zero to about 1.0 weight percent of Zr at least one element selected from the group consisting of about 0.25 weight percent to about 1.0 weight percent of Ag and about 0.1 weight percent to about 1.0 weight percent of Ce; optionally from about 1.0 weight percent to about 4.0 weight percent of Sr; optionally from about 1.0 weight percent to about 9.0 weight percent of A; optionally from about 0.1 weight percent to about 1.0 weight percent of Mn; and a balance of Mg and impurities due to production, based on total weight of the metal alloy; and methods of making the alloy;
including from about 0.5 weight percent to about 4.0 weight percent of Y, from greater than zero to about 1.0 weight percent of Ca, from about 0.25 weight percent to about 1.0 weight percent of Ag, from about 0.25 weight percent to about 1.0 weight percent of Zr, and a balance of Mg, based on total weight of the composition;
including from about 0.5 weight percent to about 4.0 weight percent of Y, from greater than zero to about 1.0 weight percent of Ca, from about 0.1 weight percent to about 1.0 weight percent of Ce, from about 0.25 weight percent to about 1.0 weight percent of Zr, and a balance of Mg, based on total weight of the composition;
including from about 0.5 weight percent to about 4.0 weight percent of Y, from greater than zero to about 1.0 weight percent of Ca, from about 0.25 weight percent to about 1.0 weight percent of Ag, from about 0.1 weight percent to about 1.0 weight percent of Ce, from about 0.25 weight percent to about 1.0 weight percent of Zr, and a balance of Mg, based on total weight of the composition;
including from about 1.0 to about 6.0 weight percent of Zn, from about 0.25 to about 1 weight percent of Ag, from greater than zero to about 1.0 weight percent of Zr, and a balance of Mg, based on total weight of the composition;
including from about 1.0 to about 6.0 weight percent of Zn, from about 0.1 to about 1 weight percent of Ce, from greater than zero to about 1.0 weight percent of Zr, and a balance of Mg, based on total weight of the composition; or
including from about 1.0 to about 6.0 weight percent of Zn, from about 0.25 to about 1 weight percent of Ag, from about 0.1 to about 1 weight percent of Ce, from greater than zero to about 1.0 weight percent of Zr, and a balance of Mg, based on total weight of the composition.

In non-limiting embodiments or aspects, the Mg alloy is devoid of Zn and Al. In non-limiting embodiments or aspects, the Mg alloy is devoid of Al. In non-limiting embodiments or aspects, the Mg alloy contains an amount of Zn and/or an amount of Al that is such as to maintain the toxicity levels of the compositions within acceptable limits.

It is understood that physical processing of alloys, such as Mg alloys, will affect physical properties of the alloys. Processing variations, such as wrought, cast, cold-rolled, hot-rolled, forged, tempered, and annealed products, as they are understood in the metallurgical arts, will yield products with different physical attributes. For example, despite having essentially the same chemical composition, T5 and T6 tempering of hot-rolled WE43 alloy will produce different hardness and ductility, among other properties. Further, casting of the same chemical composition will yield very different physical properties (See, e.g., Yu et al., Effect of T5 and T6 Tempers on a Hot-Rolled WE43 Magnesium Alloy, Materials Transactions 49(8) (2008) 1818-1821).

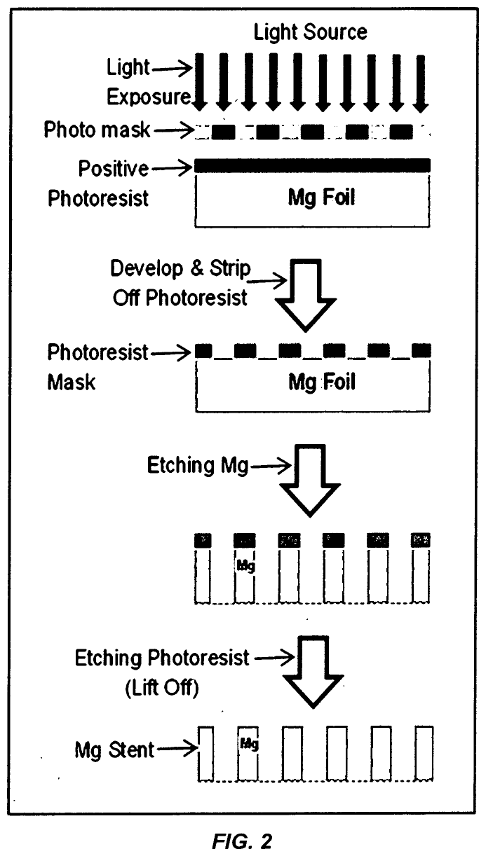

Any suitable method may be used to manufacture the frame of the prosthetic heart valve device. Due to the large diameter (e.g., in the range of 15 mm to 20 mm), the metallic, e.g. Mg, frame may not be capable of manufacture using conventional laser cutting of metallic tubing, as with conventional stent. Certain biodegradable metals, such as Mg, Zn, or alloys thereof, are not as ductile, malleable, or otherwise formable as non-biodegradable metals such as stainless steels or nitinol, as used in cardiovascular stents. As such, it may be difficult to machine or draw tubing with such a diameter and uniform thickness of the wall of, for example and without limitation, from 200 microns (200μ) to 250μ. As such, a photo-chemical etching method, e.g., as described below, may be used to etch a metallic sheet, e.g., a Mg alloy sheet, e.g., with uniform thickness of from 200μ to 250μ, rolled into a cylinder, and laser-welded with any desirable diameter, for example and without limitation, ranging from 15 mm to 25 mm, including 20 mm. See, e.g., FIG. 2, and U.S. Pat. No. 9,655,752, the disclosure of which is incorporated herein by reference, describing an exemplary photochemical etching method for Mg foil.

As described above, the device described herein also comprises a biocompatible, biodegradable polymeric heart valve. In non-limiting embodiments or aspects, the polymeric heart valve acts as a tissue scaffold that generally comprises two portions (see, e.g., International Patent Publication No. WO 2016/138416 depicting exemplary multi-leaflet valves and methods of making the valves, incorporated herein by reference). A portion of the valve is annular (forming a ring, but not necessarily defining any particular geometric shape such as a perfect circle or cylinder), and is provided as a point of attachment of the polymeric heart valve to the frame and, when implanted in a patient to tissue surrounding the device, such as the annulus of a heart valve. For instance, when the device is placed in a heart, the annular portion is configured to abut against native heart valve tissue and/or surrounding tissue when implanted, and optionally providing a suturing and anchoring structure for attachment to the frame or to native tissue, as well as providing an aperture for blood flow through the valve structure. The second portion comprises two or more flexible, coaptating leaflets that are movable relative to the first annular portion between an open configuration in which the leaflet permits blood flow through the aperture in a first direction, and a closed configuration in which the leaflet restricts blood flow through the aperture in a second direction, opposite the first direction. The leaflets may be joined with adjacent leaflets at a portion of their edges immediately adjacent to the support portion to form a commissure, and, in non-limiting embodiments or aspects, are not joined at a portion distal to the support portion, to permit blood to flow through the valve when it is open. When the valve is closed, the leaflets may be concave, meaning that the concavity extends generally towards a central axis of the aperture of the annular portion, and the leaflets contact or coaptate with adjacent leaflets to form a seal. Unless indicated otherwise, in reference to heart-valve structures described herein, concave means curved or extending towards the rotational, longitudinal, or central axis, and convex, means curved or extending outwards away from the rotational, longitudinal, or central axis. The terms coaptating, commissure, valve, and leaflet are in reference to, and generally are configured to mimic similar structures in native heart valves. In non-limiting embodiments or aspects, as noted above, the polymeric heart valve, though the shapes, orientations, and sizes disclosed herein, is adapted to provide a scaffolding for generation of nascent heart valve tissue from cells that infiltrate (or, in certain non-limiting embodiments are pre-seeded on the device) and over time, replace the biodegradable matrix of the valve structure.

A number of biocompatible, biodegradable elastomeric polymers and (co)polymers are known and have been established as useful in preparing cell growth matrices, and are useful in preparation of the polymeric heart valve described herein. Non-limiting examples of a bioerodible polymer useful in the devices described herein, include: a polyurethane, a polyester, a polyester-containing copolymer, a polyanhydride, a polyanhydride-containing copolymer, a polyorthoester, and a polyorthoester-containing copolymer. In one aspect, the polyester or polyester-containing copolymer is a poly(lactic-co-glycolic) acid (PLGA) copolymer. In other aspects, the bioerodible polymer is selected from the group consisting of poly(lactic acid) (PLA); poly(trimethylene carbonate) (PTMC); poly(caprolactone) (PCL); poly(glycolic acid) (PGA); poly(glycolide-co-trimethylenecarbonate) (PGTMC); poly(L-lactide-co-glycolide) (PLGA); polyethylene-glycol (PEG-) containing block copolymers; and polyphosphazenes. Additional bioerodible, biocompatible polymers include: a poly(ester urethane) urea (PEUU); poly(ether ester urethane)urea (PEEUU); poly(ester carbonate)urethane urea (PECUU); poly(carbonate)urethane urea (PCUU); a polyurethane; a polyester; a polymer comprising monomers derived from alpha-hydroxy acids such as: polylactide, poly(lactide-co-glycolide), poly(L-actide-co-caprolactone), polyglycolic acid, poly(dl-lactide-co-glycolide), and/or poly(I-lactide-co-dl-lactide); a polymer comprising monomers derived from esters including polyhydroxybutyrate, polyhydroxyvalerate, polydioxanone, and/or polyglactin; a polymer comprising monomers derived from lactones including polycaprolactone; or a polymer comprising monomers derived from carbonates including polycarbonate, polyglyconate, poly(glycolide-co-trimethylene carbonate), or poly(glycolide-co-trimethylene carbonate-co-dioxanone). Those of skill will appreciate that biocompatible, biodegradable, elastomeric materials may be useful for heart valves as described herein.

In aspects, diamines and diols are useful building blocks for preparing the (co)polymer compositions described herein. Diamines as described above have the structure H₂N—R—NH₂ where “R” is an aliphatic or aromatic hydrocarbon or a hydrocarbon comprising aromatic and aliphatic regions. The hydrocarbon may be linear or branched. Examples of useful diamines are putrescine (R=butylene) and cadaverine (R=pentylene). Useful diols include polycaprolactone, multi-block copolymers, such as polycaprolactone-PEG copolymers, including polycaprolactone-b-polyethylene glycol-b-polycaprolactone triblock copolymers of varying sizes. Other building blocks for useful diols include, without limitation, glycolides (e.g., polyglycolic acid (PGA)), lactides, dioxanones, and trimethylene carbonates. Diisocyanates have the general structure OCN—R—NCO, where “R” is an aliphatic or aromatic hydrocarbon or a hydrocarbon comprising aromatic and aliphatic regions. The hydrocarbon may be linear or branched.

In a one aspect, the polymer composition comprises a biodegradable poly(ester urethane) urea (PEUU), poly(ether ester urethane)urea (PEEUU), poly(ester carbonate)urethane urea (PECUU), or poly(carbonate)urethane urea (PCUU). In some examples, the composition comprises poly(ester-urethane)urea (PEUU). PEUU can be synthesized using putrescine as a chain extender and a two-step solvent synthesis method. For example, a poly(ester urethane) urea elastomer (PEUU) may be made from polycaprolactonediol and 1,4-diisocyanatobutane, with a diamine, such as putrescine as the chain extender. A suitable PEUU polymer may be made by a two-step polymerization process whereby polycaprolactone diol, 1,4-diisocyanatobutane, and putrescine are combined in a 1:2:1 molar ratio though virtually any molar feed ratio may suffice so long as the molar ratio of each monomer component is >0. In one aspect, the molar feed ratio of polycaprolactone diol plus putrescine is equal to that of diisocyanatobutane. A poly(ether ester urethane) urea elastomer (PEEUU) may be made by reacting polycaprolactone-b-polyethylene glycol-b-polycaprolactone triblock copolymers with 1,4-diisocyanatobutane and putrescine. In one aspect, PEEUU is obtained by a two-step reaction using a 2:1:1 reactant stoichiometry of 1,4-diisocyanatobutane:triblock copolymer:putrescine.

In another aspect, the composition comprises a poly(ester carbonate urethane)urea (PECUU) or a poly(carbonate)urethane urea (PCUU) material. PECUU and PCUU are described, for example, in Hong et al., Tailoring the degradation kinetics of poly(ester carbonate urethane)urea thermoplastic elastomers for tissue engineering scaffolds, Biomaterials (2010) 31(15): 4249-58. PECUU is synthesized, for example, using a blended soft segment of polycaprolactone (PCL) and poly(1,6-hexamethylene carbonate) (PHC) and a hard segment of 1,4-diisocyanatobutane (BDI) with chain extension by putrescine. Different molar ratios of PCL and PHC can be used to achieve different physical characteristics. Putrescine is used as a chain extender by a two-step solvent synthesis method. In one example, the (PCL+PHC):BDI:putrescine molar ratio is defined as 1:2:1.

The valve portion of the device described herein can be formed by any useful method, for example, by solvent casting in a mold, for example with particulate leaching to produce a porous structure, or by 3D printing or dry spinning methods, or by electrodeposition. In one aspect, the structure is cut from a polymeric mesh comprising synthetic and/or natural (e.g., ECM) polymer compositions. In one aspect, for illustrative purposes, a polymeric mesh is electrodeposited, e.g., electrospun onto a target, such as a mandrel, and the resultant structure is shaped, e.g., by cutting, into shapes such as heart valve leaflet shapes or annular shaped (see, for example, U.S. Pat. Nos. 8,535,719 B2 and 9,237,945 B2, and United States Patent Application Publication No. 2014/0377213 A1, each of which is incorporated herein by reference in its entirety for their disclosure of electrospinning methods, and variations on electrospun matrices, including synthetic and natural components). While the polymeric mesh may be isotropic, the nature of heart valve leaflet and annulus ECM often is, in part, anisotropic, and as such, the polymeric matrix that is used to prepare the heart valve may be deposited in an oriented manner, and is therefore anisotropic. Electrospinning and electrodeposition methods are broadly-known, and in electrodeposition, relative movement of the nozzles/spinnerets and target surface, e.g., by deposition onto a rotating mandrel, during electrodeposition can be used to produce an oriented pattern of fibers. As is further broadly-known, more than one polymer composition can be electrodeposited concurrently, or in a desired order, to create a layered structure. Further, solutions comprising other polymers, ECM materials (e.g., ECM gel, or solubilized ECM), cell-culture medium, cells, such as stem cells including MSCs or ASCs, blood products, therapeutic agents, can be electrosprayed onto, or into the formed fiber structure, with variable deposition timing to create optimal layering or release of the soluble fraction. The properties of electrospun elastomeric matrices can be tailored by varying the electrospinning conditions. For example, when the biased target is relatively close to the orifice, the resulting electrospun mesh tends to contain unevenly thick fibers, such that some areas of the fiber have a “bead-like” appearance. However, as the biased target is moved further away from the orifice, the fibers of the non-woven mesh tend to be more uniform in thickness. Moreover, the biased target can be moved relative to the orifice. In certain embodiments, the biased target is moved back and forth in a regular, periodic fashion, such that fibers of the non-woven mesh are substantially parallel to each other. When this is the case, the resulting non-woven mesh may have a higher resistance to strain in the direction parallel to the fibers, compared to the direction perpendicular to the fibers. In other embodiments, the biased target is moved randomly relative to the orifice, so that the resistance to strain in the plane of the non-woven mesh is isotropic. The target can also be a rotating mandrel. In this case, the properties of the non-woven mesh may be changed by varying the speed of rotation. The properties of the electrospun elastomeric scaffold may also be varied by changing the magnitude of the voltages applied to the electrospinning system.

Electrospinning may be performed using two or more nozzles, wherein each nozzle is a source of a different polymer solution. The nozzles may be biased with different biases or the same bias in order to tailor the physical and chemical properties of the resulting non-woven polymeric mesh. Additionally, many different targets may be used. In addition to a flat, plate-like target, a mandrel may be used as a target. When the electrospinning is to be performed using a polymer suspension, the concentration of the polymeric component in the suspension can also be varied to modify the physical properties of the elastomeric scaffold. For example, when the polymeric component is present at relatively low concentration, the resulting fibers of the electrospun non-woven mesh have a smaller diameter than when the polymeric component is present at relatively high concentration. One skilled in the art can adjust polymer concentrations to obtain fibers of desired characteristics. Useful ranges of concentrations for the polymer component include from about 1% wt. to about 15% wt., from about 4% wt. to about 10% wt., and from about 6% wt. to about 8% wt.

In electrospinning, polymer fibers are often deposited about the circumference of a mandrel and to generate a planar or substantially planar structure, the electrodeposited mat/matrix is cut substantially in the direction of the rotational axis of the mandrel, or in any manner to generate a useful topology, such as the shape of a heart valve, a heart valve leaflet, or portion thereof. In use, more than one electrospun mats/matrices can be attached by any useful means, such as by “sewing” using sutures, heat annealing, chemical annealing/cross-linking, etc., though it should be recognized that the method of attaching the two or more mats/matrices would have to be strong enough for the end use, e.g., to resist breakage, rupture, or herniation.

Although any form of spraying is expected to be effective, liquid, e.g., cell growth media, extracellular matrix (ECM) pre-gel, cells, a blood product, such as serum, plasma, or platelet-rich plasma, or a therapeutic composition, such as the soluble PCL ECM fraction described herein, may be electrosprayed. Electrospraying can be done before, after, or concurrently (intermittently or continuously) with the electrodeposition of polymer fibers, and is conducted in an essentially identical manner.

The composition and structures according to any aspect described herein can also include additional components, such as an active agent, such as, without limitation, one or more of an antiseptic, an antibiotic, an analgesic, an anesthetic, a chemotherapeutic agent, an anti-inflammatory agent, a metabolite, a cytokine, a chemoattractant, a hormone, a steroid, a protein, or a nucleic acid, e.g., as described above, extracellular matrix (ECM) material(s), and/or cells. Such additions that may be incorporated, by themselves, or in combination with a suitable excipient, into the compositions described herein are described below.

### ECM

The polymeric heart valve matrix described herein optionally comprises, in one aspect an extracellular matrix-derived (ECM) material, such as a gel (see, e.g., U.S. Pat. Nos. 8,361,503, and 8,691,276, the disclosures of which are incorporated herein by reference), as a base material in a polymeric composition/matrix (e.g., a composite), and/or a coating.

An “ECM material,” is a decellularized and/or devitalized material comprising or prepared from an extracellular matrix-containing tissue, and, in some non-limiting embodiments or aspects, does not solely consist of a single, isolated and purified ECM component, such as a purified collagen preparation. In some non-limiting embodiments or aspects, a single, or more than one, component of ECM is included in a polymeric matrix as described herein. Any type of tissue-derived material can be used to produce the ECM materials in the methods, compositions, and devices as described herein (see generally, U.S. Pat. Nos. 4,902,508; 4,956,178; 5,281,422; 5,352,463; 5,372,821; 5,554,389; 5,573,784; 5,645,860; 5,711,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666). The ECM material may be protease-solubilized, or otherwise-solubilized ECM material, such as ECM material that is acid-protease solubilized in acidic conditions—producing a reverse-gelling composition. In certain aspects, the ECM material is isolated from a vertebrate animal, for example and without limitation, from a mammal, including, but not limited to, human, monkey, pig, cow, and sheep. The ECM material can be prepared from any organ or tissue, including, without limitation, heart, urinary bladder, intestine, liver, esophagus, blood vessel, liver, nerve or brain, and/or dermis.

### Therapeutic Agents

In certain aspects, as described briefly above, the polymer of the heart valve can be a polymeric matrix. In non-limiting embodiments or aspects, a polymeric matrix of the heart valve comprises one or more therapeutic agents. For example, at least one therapeutic agent is added to the polymer composition described herein before it is implanted in the patient or otherwise administered to the patient. Generally, the therapeutic agents include any substance that can be coated on, embedded into, absorbed into, adsorbed to, or otherwise attached to or incorporated onto or into the polymeric heart valve described herein. Non-limiting examples of such therapeutic agents include: anti-thrombogenic agents, growth factors, chemoattractants, cytokines, antimicrobial agents, emollients, retinoids, and steroids. Each therapeutic agent may be used alone or in combination with other therapeutic agents.

Active agents that may be incorporated into the device described herein include, without limitation, anti-inflammatories, such as, without limitation, nitro-fatty acids NSAIDs (non-steroidal anti-inflammatory drugs) such as salicylic acid, indomethacin, sodium indomethacin trihydrate, salicylamide, naproxen, colchicine, fenoprofen, sulindac, diflunisal, diclofenac, indoprofen sodium salicylamide, anti-inflammatory cytokines, and anti-inflammatory proteins or steroidal anti-inflammatory agents); antibiotics; anticlotting factors such as heparin, Pebac, enoxaprin, aspirin, hirudin, plavix, bivalirudin, prasugrel, idraparinux, warfarin, coumadin, clopidogrel, PPACK, GGACK, tissue plasminogen activator, urokinase, and streptokinase; growth factors. Other active agents include, without limitation: (1) immunosuppressants; glucocorticoids such as hydrocortisone, betamethisone, dexamethasone, flumethasone, isoflupredone, methylprednisolone, prednisone, prednisolone, and triamcinolone acetonide; (2) antibodies; (3) drugs acting on immunophilins, such as cyclosporine, zotarolimus, everolimus, tacrolimus, and sirolimus (rapamycin), interferons, TNF binding proteins; (4) taxanes, such as paclitaxel and docetaxel; statins, such as atorvastatin, lovastatin, simvastatin, pravastatin, fluvastatin, and rosuvastatin; (5) nitric oxide donors or precursors, such as, without limitation, Angeli's Salt, L-Arginine, Free Base, Diethylamine NONOate, Diethylamine NONOate/AM, Glyco-SNAP-1, Glyco-SNAP-2, S-Nitroso-N-acetylpenicillamine, S-Nitrosoglutathione, NOC-5, NOC-7, NOC-9, NOC-12, NOC-18, NOR-1, NOR-3, SIN-1, Hydrochloride, Sodium Nitroprusside, Dihydrate, Spermine NONOate, Streptozotocin; and (6) antibiotics, such as, without limitation: acyclovir, afloxacin, ampicillin, amphotericin B, atovaquone, azithromycin, ciprofloxacin, clarithromycin, clindamycin, clofazimine, dapsone, diclazaril, doxycycline, erythromycin, ethambutol, fluconazole, fluoroquinolones, foscamet, ganciclovir, gentamicin, iatroconazole, isoniazid, ketoconazole, levofloxacin, lincomycin, miconazole, neomycin, norfloxacin, ofloxacin, paromomycin, penicillin, pentamidine, polymixin B, pyrazinamide, pyrimethamine, rifabutin, rifampin, sparfloxacin, streptomycin, sulfadiazine, tetracycline, tobramycin, trifluorouridine, trimethoprim sulfate, Zn-pyrithione, ciprofloxacin, norfloxacin, afloxacin, levofloxacin, gentamicin, tobramycin, neomycin, erythromycin, trimethoprim sulfate, polymixin B, and silver salts such as chloride, bromide, iodide, and periodate.

Other drugs, active agents, or compositions that may promote wound healing and/or tissue regeneration may also be included in any material described herein.

Pharmaceutically acceptable salts or prodrugs of any active agent (e.g., therapeutic agent or drug), bound to or otherwise combined with, or incorporated into the composition according to any aspect herein, may also be employed. Pharmaceutically acceptable salts are, because their solubility in water is greater than that of the initial or basic compounds, particularly suitable for medical applications. These salts have a pharmaceutically acceptable anion or cation. Suitable pharmaceutically acceptable acid addition salts of the compounds of the invention include, without limitation, salts of inorganic acids such as hydrochloric acid, hydrobromic, phosphoric, metaphosphoric, nitric and sulfuric acid, and of organic acids such as, for example, acetic acid, benzenesulfonic, benzoic, citric, ethanesulfonic, fumaric, gluconic, glycolic, isethionic, lactic, lactobionic, maleic, malic, methanesulfonic, succinic, p-toluenesulfonic, and tartaric acid. Suitable pharmaceutically acceptable basic salts include without limitation, ammonium salts, alkali metal salts (such as sodium and potassium salts), alkaline earth metal salts (such as Mg and calcium salts), and salts of trometamol (2-amino-2-hydroxymethyl-1,3-propanediol), diethanolamine, lysine or ethylenediamine. Pharmaceutically acceptable salts may be prepared from parent compounds by any useful method, as are well known in the chemistry and pharmaceutical arts.

Any useful cytokine or chemoattractant can be mixed into, mixed with, or otherwise combined with any composition as described herein. For example and without limitation, useful components include growth factors, interferons, interleukins, chemokines, monokines, hormones, and angiogenic factors. In certain non-limiting aspects, the therapeutic agent is a growth factor, such as a neurotrophic or angiogenic factor, which optionally may be prepared using recombinant techniques. Non-limiting examples of growth factors include basic fibroblast growth factor (bFGF), acidic fibroblast growth factor (aFGF), vascular endothelial growth factor (VEGF), hepatocyte growth factor (HGF), insulin-like growth factors 1 and 2 (IGF-1 and IGF-2), platelet derived growth factor (PDGF), stromal derived factor 1 alpha (SDF-1 alpha), nerve growth factor (NGF), ciliary neurotrophic factor (CNTF), neurotrophin-3, neurotrophin-4, neurotrophin-5, pleiotrophin protein (neurite growth-promoting factor 1), midkine protein (neurite growth-promoting factor 2), brain-derived neurotrophic factor (BDNF), tumor angiogenesis factor (TAF), corticotrophin releasing factor (CRF), transforming growth factors α and β (TGF-α and TGF-β), interleukin-8 (IL-8), granulocyte-macrophage colony stimulating factor (GM-CSF), interleukins, and interferons. Commercial preparations of various growth factors, including neurotrophic and angiogenic factors, are available from R & D Systems, Minneapolis, Minn.; Biovision, Inc, Mountain View, Calif.; ProSpec-Tany TechnoGene Ltd., Rehovot, Israel; and Cell Sciences®, Canton, Mass.

### Cells

In non-limiting embodiments or aspects, cells are added to a material described herein, and/or are seeded thereon prior to introduction into a patient. Non-limiting examples of useful cells include: stem cells, progenitor cells, and differentiated cells; recombinant cells; cardiac valve cells and precursors thereof; mesenchymal progenitor or stem cells; endothelial cells; or valvular interstitial cells, including, without limitation, adipose-derived, placental-derived, umbilical cord derived, bone marrow derived, circulating (blood) derived, or skeletal muscle derived progenitor cells. Further examples of potentially useful cells include: venous and arterial (e.g. radial artery) endothelial cells, endothelial progenitor cells (EPC), mesenchymal stem cells derived EC isolated from a bone marrow biopsy or human umbilical cord-derived fibroblasts, and endothelial progenitor cells (See, e.g., Siepe et al., Stem Cells Used for Cardiovascular Tissue Engineering, European Journal of Cardio-thoracic Surgery 34 (2008) 242-247). In one aspect, the cells are autologous with respect to the patient to be treated. In another aspect, the cells are allogeneic with respect to the patient to be treated.

Cells, e.g., a patient's (autologous) cells or allogeneic cells, may be pre-deposited onto the matrix, and cultured ex vivo in a suitable bioreactor or culture vessel, as are known in the cell and tissue culture fields. A patient's blood or blood cells may be deposited onto the matrix, including suitable progenitor cells. Alternatively, or in conjunction with the ex vivo culturing, the device is implanted, and by virtue of contact with circulating blood cells and adjacent tissue, such as valve annulus tissue, the polymeric heart valve is infiltrated with and populated by the patient's cells. Heart valve leaflets have been generated in vivo in animals using single polymeric (PEUU, PCUU or PECUU) heart valve leaflet matrices sewn into heart valves.

In a further aspect, an annuloplasty device, and an annuloplasty method, are provided. The ring comprises an annular biodegradable metal ring or c-shaped metallic member and a polymeric mesh, such as a biodegradable polymeric matrix, such as a non-woven mesh, e.g., an electrospun mesh, covering the metal ring or c-shaped member, for use in affixing the ring or c-shaped member in place about an annulus of a heart valve, and holding the metal ring or c-shaped member in place as it degrades. The metallic ring or c-shaped member degrades over time, e.g., over a number of months, for example, and without limitation, over one to six months, and the bioerodible mesh covering the ring or c-shaped member slowly degrades over a similar time period. The composition of the metal ring or c-shaped member and the polymer matrix may be selected and tailored so that the ring is held in place until it degrades, and prior to the degradation of the polymer matrix, so that the ring or c-shaped member is not released before it dissolves. That said, the composition of the polymer matrix may be selected to degrade prior to complete degradation of the ring or c-shaped member if there is integration of the ring or c-shaped member into tissue and no risk of its release prior to its degradation. One of ordinary skill can tailor degradation of the materials of the annuloplasty device. In one aspect, an annuloplasty method is provided comprising implanting an annuloplasty device as described in or about an annulus of a patient's heart valve for treatment of valve defects or injury.

In non-limiting embodiments or aspects, any device described herein, or portion thereof, such as the frame and/or polymeric heart valve, may be coated with a coating of a useful material. In non-limiting embodiments or aspects, the coating is a polymeric coating that slows the degradation of the frame and/or heart valve. In non-limiting embodiments or aspects, the coating is a biocompatible, non-biodegradable coating, and is optionally water-permeable or water-impermeable. In non-limiting embodiments or aspects, the non-biodegradable coating is a poly(p-xylylene) polymer, available under the trade name PARYLENE. In non-limiting embodiments or aspects, the poly(p-xylylene) polymer is one or more of PARYLENE C, PARYLENE N, PARYLENE D, PARYLENE F, and/or halogen-free PARYLENE (e.g., PARYFREE®). In non-limiting embodiments or aspects, the non-biodegradable coating is applied at a thickness that provides sufficient porosity or permeability to permit contact between the underlying frame/valve and bio-fluids, and/or factors present within the patient and therefore permit degradation of the underlying biodegradable metal or polymer. For example, and without limitation, the non-biodegradable coating may be applied at a thickness of 10 nm-10 μm, 80 nm-300 nm, or 80 nm-100 nm, all subranges therebetween inclusive, e.g., by chemical vapor deposition (CVD), or other suitable methods.

In non-limiting embodiments or aspects, the coating is a biocompatible, biodegradable coating. In non-limiting embodiments or aspects, the biodegradable coating is a polymeric coating, for example a biocompatible, elastomeric, biodegradable coating formed of one or more polymers described herein as useful for the heart valve material. In non-limiting embodiments or aspects, the biodegradable coating includes PCL and/or PGLA. In non-limiting embodiments or aspects, the biodegradable coating is applied at a thickness of up to 10 μm.

As described above, other coatings are envisioned and can be included, such as an ECM-based or containing coating, a cell-containing coating, and/or a therapeutic agent (in a matrix or otherwise). In non-limiting embodiments or aspects, the coating is an antithrombogenic coating, e.g., the coating is anti-thrombogenic or includes one or more anti-thrombogenic compositions or moieties. In one aspect, the antithrombogenic coating is a polyurethane polymer composition comprising, integrated into the polymer backbone, into a crosslinker, or pendant from a polymer backbone an antithrombogenic group, such as, without limitation, a zwitterion, a phosphorylcholine or a betaine, such as trimethylglycine, a sulfobetaine, or a carboxybetaine, for example and without limitation, as described in U.S. Pat. No. 9,808,560, the disclosure of which is incorporated herein by reference in its entirety. In other aspects, the device or a portion thereof is coated with heparin, or modified with hydrophilic surface groups such as polymers or multimers of ethylene glycol.

In use, the devices described herein are surgically-implanted, e.g., within the annulus of a heart valve. Methods of implanting the structures are known in the art and are a matter of using standard surgical techniques, such as for biasing a prosthetic heart valve in place using a balloon catheter, or for sewing an annuloplasty ring in place.

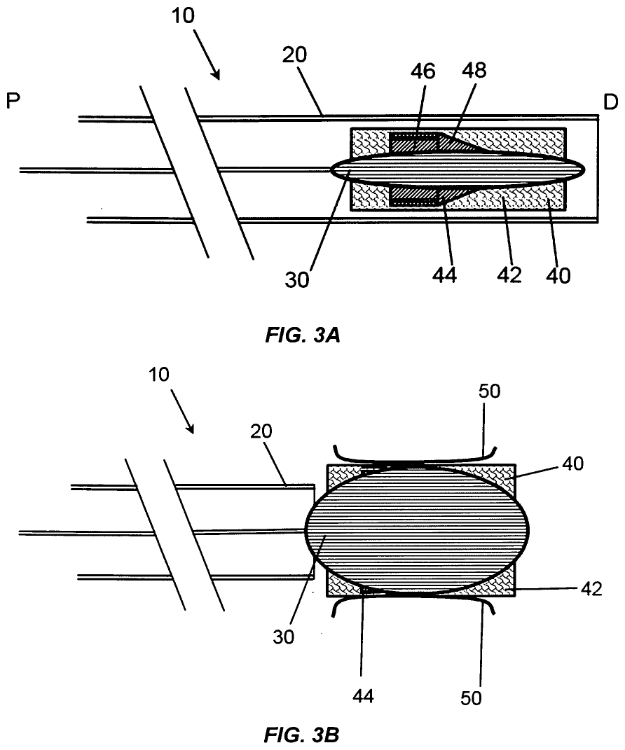

In aspects, the heart valve device is deployed percutaneously, e.g., by the Seldinger technique using a catheter. A typical catheter 10 is depicted in FIGS. 3A and 3B, and comprises a sheath 20 having a proximal end P and a distal end D of a diameter that fits within a patient's vasculature, e.g., 15 French and narrower, though in some instances where access is through a large blood vessel, larger, and within the sheath one or more guide wires for guiding the distal end of the catheter through a patient's vasculature. Within the distal end of the sheath 20 is an inflatable balloon 30 that can be pushed beyond the distal end of the sheath and inflated (see, FIG. 3B). Also within the sheath 20, surrounding the balloon 30, is an expandable heart valve device 40, e.g., as described herein, comprising a cylindrical frame 42 depicted in its compressed state in FIG. 3A, and its expanded state in FIG. 3B, and a polymeric heart valve 44, comprising an annular portion 46 and leaflets 48. The distal end D of the catheter 10 is guided to a point adjacent to a heart valve annulus, e.g., by guide wires (not shown) or any useful method. The device 40 is then pushed, along with the balloon 30, beyond the distal end D of the sheath 20, into place in a heart valve annulus 50, and once in place, the balloon 30 is expanded, thereby biasing the frame 42 against the annulus 50 of the patient's heart valve, and fixing the device 40 in place. The balloon is then deflated and retracted from the heart valve device 40, and the catheter is removed from the patient, leaving the heart valve device 40 in place in the patient's heart. Although described above in the context of a percutaneous catheter, the device depicted in FIGS. 3A and 3B may be adapted for a transapical approach, permitting use of a larger-diameter sheath (e.g., a catheter, cannula, or trocar) as compared to the sheath of the percutaneous catheter of FIGS. 3A and 3B that is used to deliver the heart valve device through, e.g., a peripheral blood vessel.

As will be appreciated by those of ordinary skill in the art, the heart valve device, e.g., the frame and polymeric heart valve, as well as the delivery catheter configuration may have any useful shape and size, for example and without limitation, the configuration of the conventional SAPIEN 3 transcatheter heart valve, SAPIEN XT transcatheter heart valve, and the NovaFlex+ and Ascendra+Systems, commercially available from Edwards Lifesciences Corporation of Irvine, Calif.

In aspects, a method of repairing an injured or damaged heart valve, such as a mitral valve, a tricuspid valve, an aortic valve, or a pulmonic valve, in a patient is provided, comprising placing the heart valve device according to any aspect, embodiment, or example herein, in the annulus of a heart valve of a patient. The device may be placed, for example, and without limitation, by percutaneous route, transapical route, by a minimally invasive route, or by open heart surgery.

### EXAMPLES

### Example 1

A transcatheter heart valve frame was made by photo-chemical etching. A conventional stented bioprosthetic valve is comprised of a radiopaque cobalt-chromium frame, trileaflet bovine pericardial tissue valve, and polyethylene terephthalate (PET) fabric skirt. Our team replaced the Co—Cr frame with an AZ31 Mg frame manufactured by photo-chemical etching. Pictures of the frame are shown in FIG. 1. Details related to the photo-chemical etching approach are provided in U.S. Pat. No. 9,655,752 (see also, FIG. 2), incorporated herein by reference in its entirety. This technique avoids use of expensive Mg based tubing and laser cutting operations. The starting material is a rectangular sheet of Mg alloy AZ31, with dimensions 200 mm×500 mm and thickness of 250 microns, purchased from Goodfellow. According to the vendor, the composition of this alloy is: 3% A, 1% Zn, and balanced with pure Mg. Any other Mg alloys can be used as well. This approach is simple and accommodates design changes and high throughput. It includes photo-lithographically transferring the frame texture on the foil, followed by chemical etching. The resulted etched foil has a desired feature that is determined by the photolithographic mask. Finally, the two-dimensional frame structure is rolled to a cylinder and the seam is laser-welded. The photochemical etching generates no residual stress in the material during the manufacturing process. Further, no post-treatment of the frame after manufacturing is required.

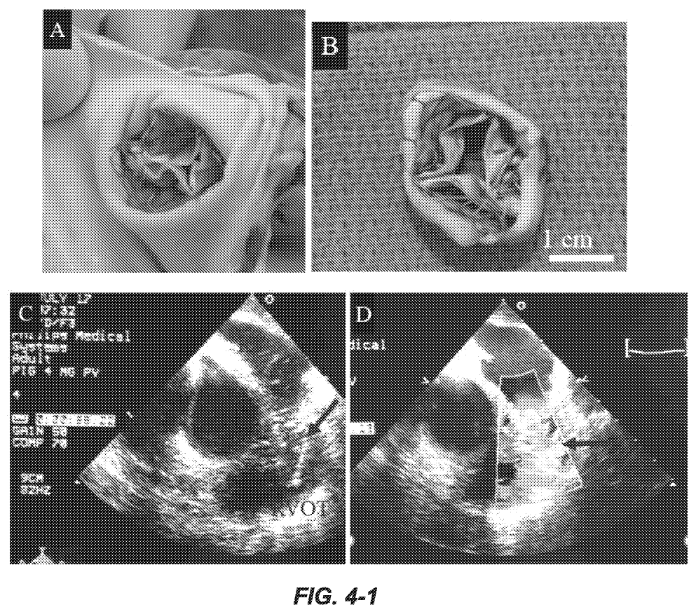

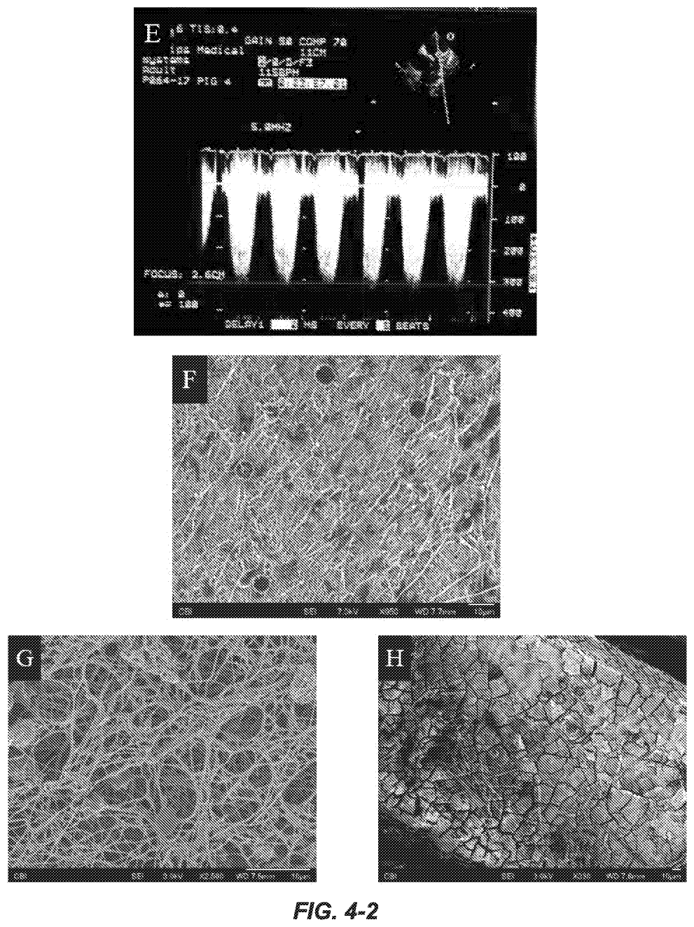

A prototype has been tested in vivo on acute (<24 hrs) porcine pulmonary replacement model (FIG. 4; see also Example 2, below). Briefly, a cylindrical frame manufactured from a sheet of AZ32 Mg alloy was prepared by photochemical etching as described above. The method includes photo-lithographically transferring the frame texture on the foil, followed by chemical etching. The resulted etched foil has a desired feature that is determined by the photolithographic mask. Finally, the two-dimensional frame structure is rolled into a cylinder and the seam is laser-welded. The photochemical etching generates no residual stress in the material during the manufacturing process. Further, no post-treatment of the frame after manufacturing is required. A trileaflet bovine pericardial tissue valve with a polyethylene terephthalate (PET) fabric skirt was attached to the frame and was successfully implanted in a pig heart. FIG. 4 further depicts results of this test.

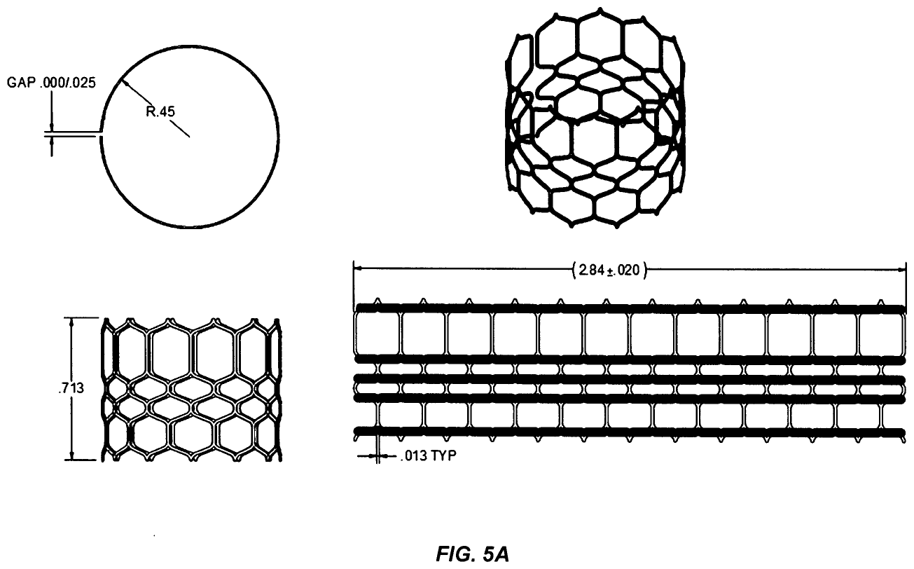

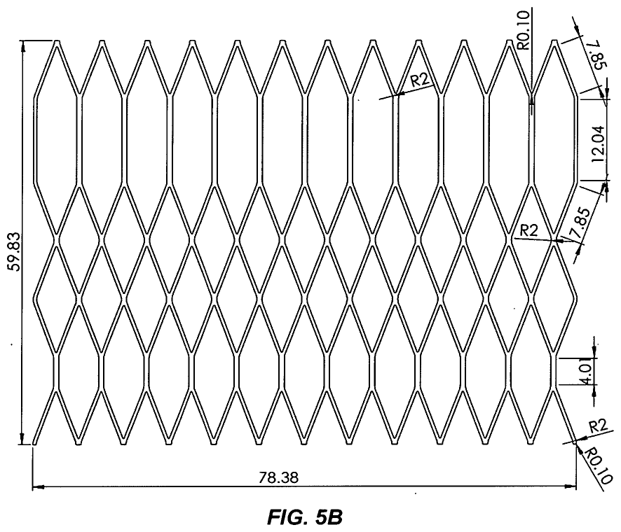

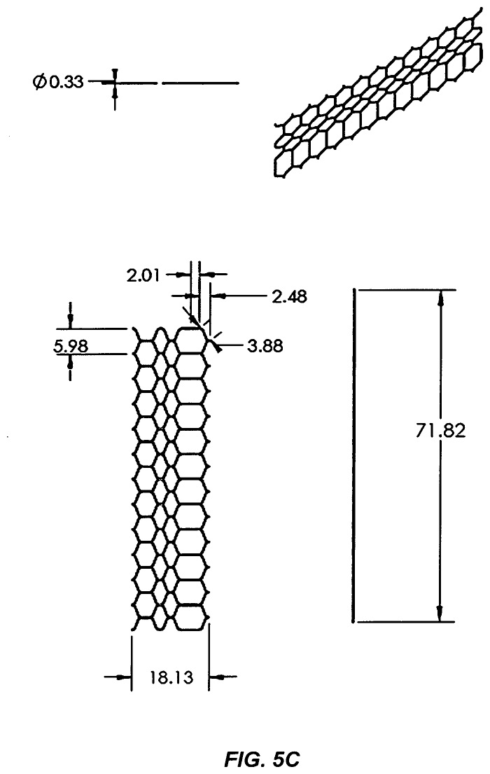

In one example, a heart valve frame was prepared from AZ31 as depicted in FIGS. 5A-5C, depicting dimensions. The heart valve frame was crimped using a blockwise manual crimper on to a mandrel of diameter 19 mm. The heart valve stent crimped and observed the changes in length and deformation of the patterns on the walls. After crimping, the heart valve stent was measured and the diameter was 19 mm; there was not much increase in the length; there were some deformations in the shape of patterns after crimping; and deformation in the shape of patterns were observed mainly in the vicinity of welding. The frame was then placed on a 19 mm stainless steel mandrel and crimped into shape.

### Example 2

Pursuit of ideal heart valve solutions aim to provide thrombosis-free durability. A scaffold-based polycarbonate urethane urea (PCUU) tissue engineered heart valve (TEHV) designed to mimic native valve microstructure and function was employed. This proof-of-concept study examined the acute in vivo function of a stented TEHV in a porcine model.

### Methods

### Ethics Statement

The study protocol was approved by the Institutional Animal Care and Use Committee of the University of Pittsburgh. Animals were cared for in accordance with the National Institutes of Health Guide for the Care and Use of Laboratory Animals.

### Fabrication of Valve Scaffold and Stented Valve Fabrication

Scaffold-based PCUU valves were fabricated and processed with a double-component deposition (DCD) technique and electrospun on a semi-lunar valve mandrel. The following electrospinning conditions were utilized: voltage gap 13 kV, gap 4.5 cm, rotational speed 200 rpm, polymer flow rate 1.5 ml/hr, mandrel major diameter 23 mm. Mg AZ31 alloy was processed using photochemical etching to create a stent that was biodegradable over time. The scaffold-based PCUU valve was mounted on the Mg stent using 5-0 polypropylene sutures distributed at six locations as illustrated (FIG. 1). The entire prosthesis was then sterilized by three serial 70% ethanol washes for 20 min, followed by three washes in phosphate-buffered saline for 20 min. The process was conducted inside a biological hood where each device was cumulatively exposed to UV light.

### Surgical Procedure

Five Yorkshire pigs were obtained and underwent pre-operative veterinary assessment and acclimatization prior to surgical procedure. Anesthesia was induced with intramuscular Ketamine, inhaled Isoflurane, and intramuscular Telazol. General anesthesia was maintained throughout the operation with titration of Isoflurane by a veterinary anesthesia team. Animals were intubated, and subsequently internal jugular venous cannulation and carotid arterial cannulation were completed via direct cutdown for monitoring and venous access. Median sternotomy was performed, and normothermic cardiopulmonary bypass was established via bi-caval venous cannulation and ascending aortic cannulation after complete systemic heparinization. Adequate heparin dosing was gauged by activated clotting time of greater than 410 sec.

The pulmonary artery was incised directly during beating-heart cardiopulmonary bypass and the native pulmonary valve was excised. The stented PCUU prosthesis was implanted using a running 4-0 polypropylene annular suture. The pulmonary artery was closed, cardiopulmonary bypass was weaned, and the animal was decannulated followed by protamine sulfate administration. Activated clotting time (ACT) was obtained after protamine administration to ensure return to baseline coagulation parameters. Epicardial 2-dimensional echocardiography was completed intraoperatively and at study endpoint/euthanasia. Animals were continuously monitored by veterinary intensive care staff for 1-12 hours post valve implant per study protocol, and vital signs and serial arterial blood gas measurements were used during the postoperative period. The initial animal was extubated, with the remaining animals being ventilated throughout the post-operative study period prophylactically to prevent isolated non-cardiac respiratory complications that had developed in the first animal. Animals underwent euthanasia at the determined study endpoint, or if a humane endpoint was reached according to study protocol.

### Prosthetic Implant Analysis

All animals underwent post-operative epicardial echocardiography to assess acute valve function after surgical implantation. Echocardiographic evaluation included valve leaflet motion and prosthetic valve positioning, continuous-wave Doppler to quantify forward flow through the TEHV as well as any potential regurgitation, and color flow Doppler to identify any turbulence or regurgitant jet of the TEHV. Epicardial echocardiography was also performed at study endpoint/euthanasia in 4 animals occurring at 1, 4, 8, and 12-hour time points to confirm valve function was unchanged.

Gross explant analysis was completed immediately after study endpoint occurred. Implants were assessed for gross degradation or damage, thrombosis, or any other malfunction. The five PCUU valves were carefully removed from the Mg alloy stents, and all underwent separate analysis. One PCUU leaflet from each valve was delegated for biaxial mechanical testing, one for histologic evaluation of early cellular infiltration, and one for scanning electron microscopy (SEM) for surface analysis. The five Mg stents also underwent SEM analysis to examine surface morphology.

Biaxial stress-strain analysis in stress control mode was performed using a customized biaxial tensile testing device. Histology was performed by fixing samples in 10% formalin for fixation and sectioning, followed by hematoxylin and eosin (H&E) staining to identify cells within the PCUU valve scaffold cross section. SEM imaging was performed on PCUU leaflets after fixation in 2% glutaraldehyde and Mg stent samples after sputter coating with Pd/Au on a standard SEM (Jeol JSM633OF).

### Statistical Methods

Numerical data are presented as mean±standard deviation. There were no comparisons nor advanced statistical methods used in this pilot study due to limited sample size.

### Results

### Surgical TEHV Implant Procedure

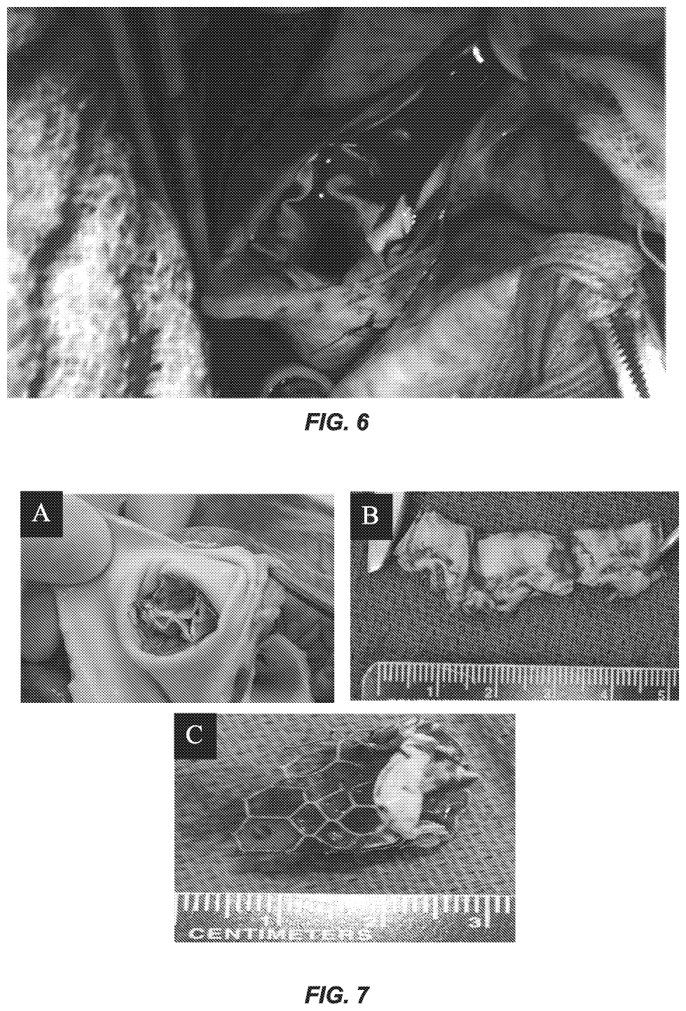

Five Yorkshire pigs, weight 89±3 kg, underwent successful TEHV placement in the described manner (FIG. 6). The PCUU sewing cuff performed well and held the polypropylene suture well. The Mg AZ31 stent body was rigid and brittle, with deformation occurring under normal surgical handling conditions. The first animal was found to have a large atrial septal defect with tricuspid valve dysplasia and was unable to be weaned from cardiopulmonary bypass after TEHV placement. The 4 subsequent animals were successfully weaned from cardiopulmonary bypass and had the chest closed. Protamine administration normalized the ACT to baseline post-operatively in all subjects. Mean cardiopulmonary bypass time was 92±19 min. Of note, each successive case had a shorter cardiopulmonary bypass time as the model developed, with the final case being 64 min.

### Epicardial Echocardiography

All animals underwent epicardial echocardiography immediately post-operatively, as well as prior to study endpoint/euthanasia to confirm valve function. The first study animal was successfully extubated, but later succumbed of non-cardiac respiratory complications with unchanged echocardiographic valve function at the pre-euthanasia 8 hour end point. The remaining animals were ventilated through study endpoints of 1 hour, 4 hours, and 12 hours respectively. The animal with the previously undiagnosed tricuspid dysplasia showed free leaflet motion and no valvular regurgitation on 1 L/min of cardiopulmonary bypass flow prior to euthanasia at the 1 hour time point for inability to wean. The remaining 4 animals were weaned completely from bypass prior to final echocardiographic evaluation. Free leaflet motion within the TEHV stent was noted universally with 2D echo, as was adequate prosthetic placement in the proximal pulmonary artery (FIG. 4C). Color flow Doppler indicated no pulmonic regurgitation, with forward non-turbulent flow through the TEHV (FIG. 4D). Continuous-wave Doppler confirmed forward flow with average peak velocity of 2.0±0.9 m/sec. There was no demonstrable regurgitation of the valve on color Doppler examination (FIG. 4E).

### Gross Explant Analysis

All valves were explanted immediately following euthanasia (FIG. 7A). Gross explant analysis showed that the PCUU leaflets were intact, without any signs of structural wear or damage. There was no thrombosis associated with the leaflets (FIG. 7B). The Mg AZ31 stents suffered multiple fracture points due to their brittle nature and normal surgical handling, yet there was no embolization noted. On two of the five stents, there was a distinct non-obstructive non-filamentous fibrin sheath formed after 6 and 12 hours (FIG. 7C).

### Explant Evaluation

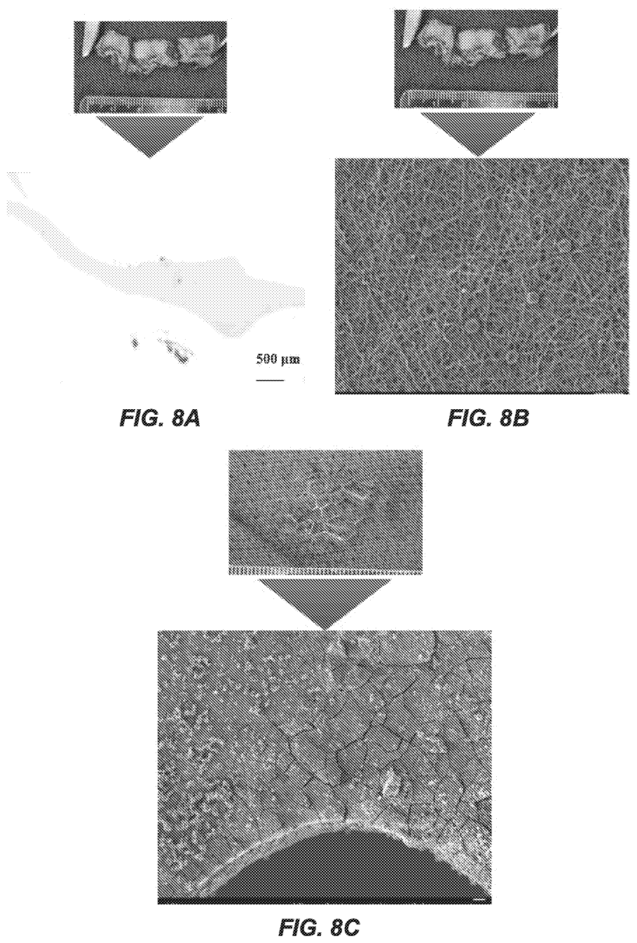

Biaxial stress-strain analysis of explants revealed no difference when compared with the characteristics of the pre-implantation sample; this element was consistent with the absence of structural damage observed by visual inspection. As expected, histological analysis of the PCUU leaflets up to 12 hours demonstrated no cellular infiltration into the TEHV leaflets, and intact PCUU leaflets without evidence of early degradation (FIG. 8A). SEM of the PCUU leaflet demonstrated intact fibrous and porous structure, with no activated platelet deposition or evidence of degradation (FIG. 8B). SEM of the Mg stent body showed expected early surface oxidation, but no evidence of bulk degradation up to 12 hours (FIG. 8C).

### DISCUSSION

This study demonstrated the acute in vivo function of a novel scaffold-based PCUU TEHV on a degradable Mg stent frame and confirmed intact histologic and non-thrombogenic ultrastructural integrity with preserved bi-axial mechanics. These findings suggest that acute biomechanical function and leaflet biocompatibility are acceptable in the pulmonary position and support chronic animal testing of a biodegradable TEHV.

However, several issues remain to be answered. These include: 1) the optimal material and its fabrication to align with natural occurring valvular function and biomechanical properties directed at durability, 2) the histologic biocompatibility to permit endothelialization followed by multi-layer valve replication directed at non-thrombogenic functional tissue to permit somatic growth, and 3) the adaptability of the technique of implantation for all positions and the potential for transcatheter application.

The concept of in situ cellularization strategies for TEHVs have been proposed as an alternative to in vitro cellularization strategies. The technique in the current study of DCD electrospinning to fabricate bioinspired PCUU heart valves leverages this paradigm. This preliminary study successfully demonstrated the capability of DCD electrospinning to reproduce a semi-lunar valve to accurate annular size and leaflet shape configuration. Previous challenges have been to ensure that scaffold creation meets the requirements of a fully functional valve prior to cell recruitment and replacement with native tissue. This study of the PCUU TEHV in the pulmonary position represents the first demonstration of this metric for a DCD trileaflet scaffold-based valve system.

An immediately bioavailable and functional scaffold-based heart valve has potential challenges with short term functional durability and histologic integrity. This study demonstrated early successful function of the PCUU TEHV in the pulmonary position via epicardial echocardiography. The acute nature of this study did not allow sufficient time for endothelization of the PCUU leaflets, as shown by histology. However, it is important to note that there was no macro or micro thrombosis of the leaflets and no platelet aggregation or inflammatory reaction via SEM up to the 12-hour endpoint. This demonstrates encouraging early leaflet biocompatibility. Further studies will need to include histochemical analysis to identify local inflammatory response, endothelial cell recruitment, stent integration in the wall, calcification, and in situ degradation profile of the polymer. These steps will be critical to identify the need for structural support duration of any biodegradable stent, such as the Mg alloy stent used in this study. Additionally, the looming questions will be the progressive functionality of the valve during this process as previous studies using TEHV leaflets demonstrate shrinkage that leads to valve failure. The polyurethane chemistry employed to generate the scaffold can readily be manipulated to control its degradation time as in vivo experience might dictate.

Implantation of a TEHV may be performed using current techniques for homograft implantation or stentless prostheses. However, providing a biodegradable stent frame that permits functional ingrowth while delivering both ease of implant and the potential for transcatheter application may provide some advantages. The current study used a Mg AZ31 alloy stent providing co-linear integrity adjacent to the root and commissures in the pulmonary or aortic position that resorbs over time as the valve potentially recruits native tissue for a support system. For this to be successful, clear degradation rates and conditions of both the individual polymeric scaffold as well as the Mg alloy stent need to be established. This is currently under study and will add greatly to the potential to combine these two technologies. The rate of Mg AZ31 degradation may be too rapid, and thus not provide support for a time window long enough to allow for sufficient endogenous tissue growth and mechanical stability of the device. Previous work has established that dip-coating of Mg stents with biodegradable polyurethanes can improve control and prolong this process, making it tailorable to TEHV leaflet degradation as applied in the current study. Therefore, polymeric coating, metallic surface alterations and profile reduction of the stent design may readily mitigate the early platelet activation and fibrin sheath formation observed in the current study. If proven to be histologically and functionally durable, the combination of a Mg stent with a PCUU TEHV, both with time-driven degradation properties through fabrication, may provide a disruptive valve solution for both congenital and adult application.

### CONCLUSIONS

This study shows that a novel biodegradable scaffold-based TEHV supported with a biodegradable Mg alloy stent functions well in an acute in vivo environment. PCUU leaflet mechanics and structure are maintained with up to 12 hours of in vivo blood contact and physiologic stress without thrombus formation. The Mg stent has evidence of rapid surface oxidation and fibrin sheath formation, which will require additional development prior to use. This proof of concept study provides a foundation for chronic studies to examine the degradation profile and in vivo tissue recruitment activity of polymeric scaffold-based heart valves.

The present invention has been described with reference to certain exemplary embodiments. However, it will be recognized by those of ordinary skill in the art that various substitutions, modifications, or combinations of any of the exemplary embodiments may be made without departing from the spirit and scope of the invention. Thus, the invention is not limited by the description of the exemplary embodiments.

1. A prosthetic heart valve device comprising:
a biocompatible and biodegradable metal frame comprising a proximal end, a distal end, and a sidewall therebetween, the sidewall comprising a plurality of openings therethrough; and
a biocompatible and biodegradable polymeric heart valve comprising an annular portion attached by at least one contact point to the proximal end of the frame and at least one leaflet attached to and extending distally from the annular portion.

2. The device of claim 1, wherein the frame comprises a magnesium (Mg) alloy, optionally comprising Al, Zn, Mn, Y, Nd, or Zr, such as an alloy selected from the group consisting of AZ31, AZ61, AZ91, or WE43.

3. (canceled)

4. (canceled)

5. (canceled)

6. (canceled)

7. (canceled)

8. (canceled)

9. The device of claim 1, wherein the biocompatible and biodegradable polymer comprises a polyurethane, such as a polycarbonate urethane urea (PCUU).

10. (canceled)

11. The device of claim 1, wherein the polymeric heart valve comprises two or three leaflets.

12. The device of claim 1, further comprising a polymeric coating on at least a portion of the device, wherein the polymeric coating optionally is a porous, non-biodegradable coating, such as a poly(p-xylylene) polymer, for example one or more of parylene C, parylene N, parylene D, parylene F, or a halogen-free parylene.

13. (canceled)

14. The device of claim 12, wherein the polymeric coating is a non-degradable coating having a thickness of 10 nm-10 μm.

15. (canceled)

16. (canceled)

17. The device of claim 12, wherein the coating is an antithrombogenic coating, optionally comprising a polyurethane with an antithrombogenic group, such as a phosphorylcholine or a betaine.

18. (canceled)

19. (canceled)

20. A method of manufacturing a heart valve device comprising attaching an annular portion of a biocompatible and biodegradable polymeric heart valve comprising an annular portion and a leaflet extending therefrom to a proximal end of a biocompatible, biodegradable metal frame having a proximal end, a distal end, and a sidewall therebetween, the sidewall comprising a plurality of openings therethrough.

21. The method of claim 20, wherein the frame comprises a Mg alloy, optionally comprising Al, Zn, Mn, Y, Nd, or Zr, such as an alloy selected from the group consisting of AZ31, AZ61, AZ91, or WE43.

22. (canceled)

23. (canceled)

24. (canceled)

25. (canceled)

26. (canceled)

27. (canceled)

28. The method of claim 20, wherein the biocompatible and biodegradable polymer comprises a polyurethane, such as a polycarbonate urethane urea (PCUU).

29. (canceled)

30. The method of claim 20, wherein the polymeric heart valve comprises two or three leaflets.

31. The method of claim 20, further comprising a polymeric coating on at least a portion of the device, wherein the polymeric coating optionally is a porous, non-biodegradable coating, such as a poly(p-xylylene) polymer, for example one or more of parylene C, parylene N, parylene D, parylene F, or a halogen-free parylene.

32. (canceled)

33. The method of claim 31, wherein the polymeric coating is a non-degradable coating having a thickness of 10 nm-10 μm.

34. (canceled)

35. (canceled)

36. The method of claim 31, wherein the coating is an antithrombogenic coating, optionally comprising a polyurethane with an antithrombogenic group, such as a phosphorylcholine or a betaine.

37. (canceled)

38. (canceled)

39. A method of repairing a heart valve in a patient, comprising placing the device of claim 1 in a heart valve annulus of the patient.

40. The method of claim 39, wherein the heart valve device is expanded in place in the heart valve annulus of the patient using a balloon catheter.

41. The method of claim 39, wherein the device is delivered percutaneously or transapically.

42. A kit comprising a catheter and the device of claim 1.

43. The device of claim 1, comprising:
an AZ31 Mg alloy, substantially cylindrical frame comprising a proximal end, a distal end, and a sidewall therebetween, the sidewall comprising a plurality of openings therethrough;
a PCUU heart valve comprising an annular portion attached by at least one contact point to the proximal end of the frame and two or three leaflets attached to and extending distally from the annular portion; and
a polymeric coating on at least a portion of the frame.

44. The device of claim 1, comprising:
a WE43 Mg alloy, substantially cylindrical frame comprising a proximal end, a distal end, and a sidewall therebetween, the sidewall comprising a plurality of openings therethrough;
a PCUU heart valve comprising an annular portion attached by at least one contact point to the proximal end of the frame and two or three leaflets attached to and extending distally from the annular portion; and
a polymeric coating on at least a portion of the frame.
